(19) United States
(12) Patent Application Publication
(10) Pub. No.: US 2011/0270040 A1
(43) Pub. Date: Nov. 3, 2011

(54) PLANNING FOR CURVATURE INTERACTIONS, MULTIPLE RADII OF CURVATURE AND ADAPTIVE NEIGHBORHOODS

(75) Inventors: Aleksandra Popovic, New York, NY (US); Karen Irene Trovato, Putnam Valley, NY (US)

(73) Assignee: KONINKLIJKE PHILIPS ELECTRONICS N.V., EINDHOVEN (NL)

(21) Appl. No.: 13/142,449

(22) PCT Filed: Nov. 10, 2009
(86) PCT No.: PCT/IB2009/054996

§ 371 Date: Jun. 28, 2011

Related U.S. Application Data
(60) Provisional application No. 61/141,119, filed on Dec. 29, 2008.

(51) Int. Cl. A61B 1/32; G06G 7/60; G06F 3/00

(52) U.S. Cl. 600/208; 710/8; 703/11

(57) ABSTRACT

Planning deployment of a medical robot based on concentric cannulas takes into account multiple radii of curvature. The radii of curvature are dependent on tube diameter. Tubes of smaller diameter can have tighter radii of curvature. Planning also takes into account moment of inertia and elasticity of tubes. For the purposes of planning, an A* algorithm is used for cost wave propagation together with a configuration space, a cost metric, and a neighborhood. The neighborhood is adaptive. The adaptive neighborhood can be different for each node in the configuration space data structure and depends on curvature affecting properties of individual tubes used to achieve a path from a most distal point to a most proximal point within a body to be examined.

The following related applications and patent documents are incorporated herein by reference:
U.S. Pat. No. 4,949,277, issued Aug. 14, 1990 to Trovato et al.
U.S. Pat. No. 5,879,303, issued Mar. 9, 1999 to Averkiou et al.
U.S. Pat. No. 6,604,005, issued Aug. 5, 2003 to Dorst et al.
Prior, co-pending US application Ser. No. 12/088,870 of Trovato et al., filed Oct. 6, 2006 (3D Path Planning, Simulation and Control System), U.S. Patent Application Publication no. 2008/0234700, Sep. 25, 2008.
Prior, co-pending U.S. provisional applications nos. 61/075,886, Jun. 26, 2008 and 61/099,223, Sep. 23, 2008, of Trovato et al. (Method and System for Fast, Precise Path Planning), which is International application no. PCT/IB2009/05250, filed Jun. 16, 2009.
Prior, co-pending U.S. provisional application No. 61/106,287 of Greenblatt et al., filed Oct. 17, 2008 (Interlocking Nested Cannula), which is International application no. PCT/IB2009/054474, filed Oct. 12, 2009.
Prior, co-pending International application no. IB2007/053253 of Trovato, filed Aug. 15, 2007 (Active Cannula Configuration for Minimally Invasive Surgery), International Publication no. WO 2008/032230 A1, Mar. 20, 2008.
Prior, co-pending U.S. Provisional application No. 61/075,401 of Trovato, filed Jun. 25, 2008 (Nested Cannulae for Minimally Invasive Surgery)), which International application no. PCT/IB2009/052521, filed Jun. 12, 2009.

These documents, when taken cumulatively, describe a medical application, which will be roughly summarized as follows:

The invention relates to the field of planning insertion of concentric cannulas into a body, for instance a human body of a medical patient.

In FIG. 1, a patient 101 is scanned in a scanning device 102. The scanning device may be of any suitable type, such as ultrasound, CT scanning, or MRI scanning. Any portion of the patient's body may be scanned, for example the lungs. The result of the scan will be to show interior structure of the patient's body. The interior structure may include tubular passages, such as the airways of a lung, blood vessels, the urethra, nasal passages or intestines. The interior spaces may be more open, such as the stomach, the bladder or the sinuses. In some cases the interior structure will be solid tissue, but where certain areas are preferred, for instance within the brain. The medical application is not limited to any particular scanning technique or any particular interior space of the body.

The scanning device will include a processor 103 for gathering and processing data from the scan. The processor may be of any suitable type and will typically include at least one machine readable medium for storing executable program code and data. There may be multiple processors and multiple storage media of one or more different types. The processor will often have some way of communicating with outside devices. This processor is illustrated with an antenna 105 for wireless communication, but the communication might equally well be wired such as to the Internet, infrared, via optical fiber, or via any suitable method. The scanning device will also include at least one user interface 104, including one or more of: a display, a touch sensitive screen, a keyboard, a pointer device, a microphone, a loudspeaker, a printer, and/or any other user interface peripheral. The invention is not limited to any particular peripherals for communicating with a user or with outside equipment.

While all processing may occur within the scanning device, there may also be an outside processor 106 for performing planning of a path, and an assumed set of ‘net shapes’ to follow the path. The processor 106 will be associated with at least one medium 107 for storing data and program code. The medium 107 may include various types of drives such as magnetic, optical, or electronic, and also memory such as cache where executing code and data structures may reside. The output of the planning process is illustrated schematically and includes a technical specification 108 in any appropriate format and also the concentric cannulas 109 themselves.

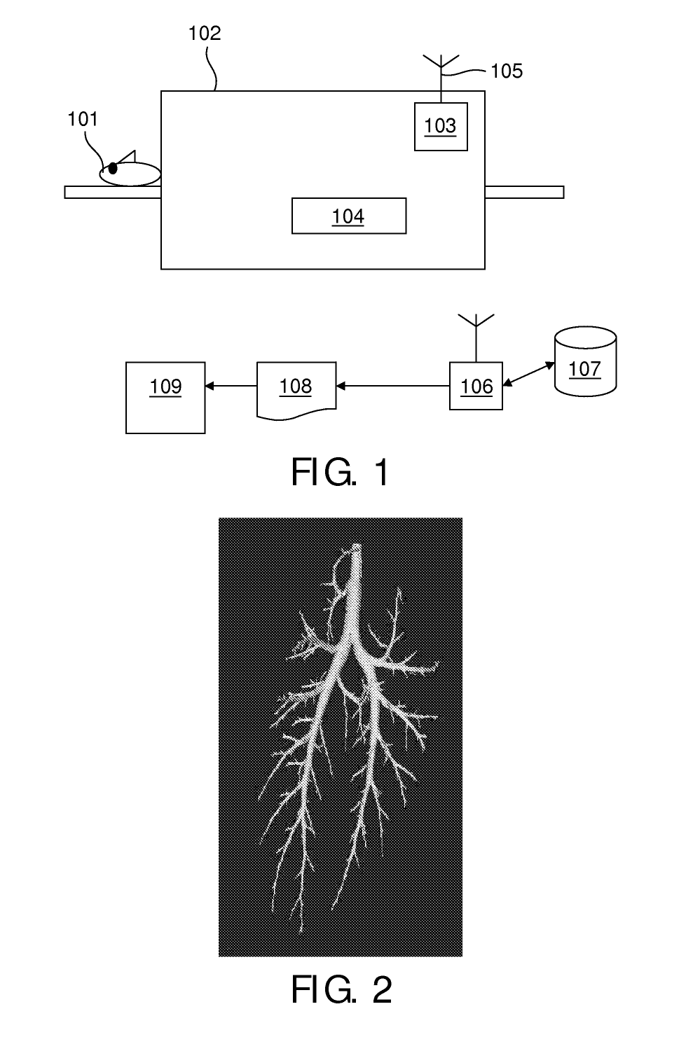

FIG. 2 shows an image of tubular passages in a patient's lungs segmented from a scan. It is desirable to insert medical devices into the tubular passages, since this minimizes damage en route to a target location. This type of surgery is called NOTES (Natural orifice translumenal endoscopic surgery) when an endoscope is used to travel through passages. This type of surgery does not require that the surgical target be within the tubular access, but rather that the target is reached with less trauma by having tools that travel through existing tubes, so that the target may be reached translumenally.

Tubular devices, such as Active Cannulas, have been proposed, see e.g. R. J. Webster et al., “Toward Active Cannulas: Miniature Snake-like Surgical Robots” 2006 IEEE/RSJ (October 2006, Beijing, China) pp. 2857-2863. These devices rely on the interaction between two or more tubes to cause lateral motion as they rotate relative to one another. As they extend from one another, they can also cause various lateral motions, particularly if they have different curvatures along a single tube. If the motion is carefully characterized, these motions can be used to reach multiple locations, similar to a robot in free space. However these devices can have difficulty when extended translumenally, if the lateral motion is greater than the available maneuver space. While the Webster article considers interactions of tubes during deployment, it lacks consideration of issues relating to making Active Cannulas follow a planned path.

Such devices may assist in gathering data, gathering tissue, or performing other procedures. Based on a patient image for example, a set of tubes can be extended, from largest to smallest so that, when deployed, they have a structure where at least a portion of each cannula will remain at the proximal end of the patient while smaller cannulas will extend into the patient interior space in reverse order of diameter. Thus the fattest cannulas will end more proximally, while the thinnest cannulas will extend more distally. Herein a cannula will be considered more distal if it ends more distally when deployed—and more proximal if it ends more proximally when deployed.

Nested Cannulas are somewhat different from Active Cannulas, since they are configured to reach specific locations in a specific environment with minimal lateral motion (wiggle). In one variety of Nested Cannula, the tubes are interlocked so that they do not rotate with respect to one another. Insertion should minimize trauma to the tubular passageways or other tissues. Such trauma can result from movements of the cannulas. Nested Cannulas are, for example, described in prior, co-pending U.S. provisional application No. 61/106,287 of Greenblatt et al., filed Oct. 17, 2008 (Interlocking Nested Cannula), which is International application no. PCT/IB2009/054474, filed Oct. 12, 2009.

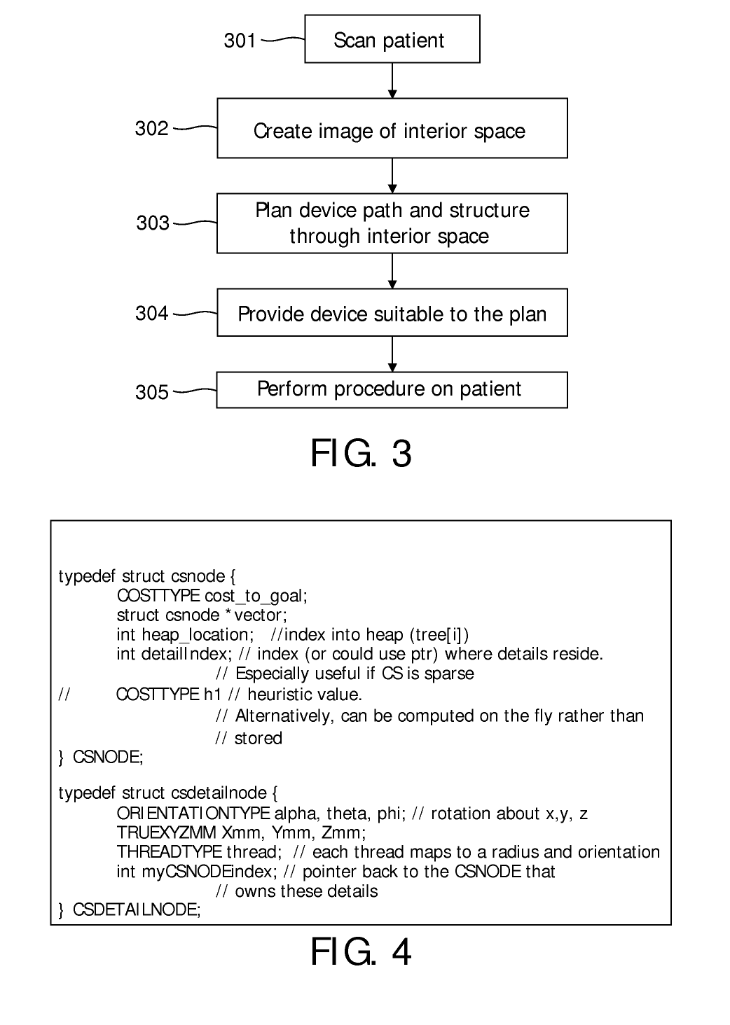

FIG. 3 shows schematically an example of the process to be followed. First, the patient is scanned at 301. An image is then created at 302 indicating forbidden regions and, typically, the costs for passing through other regions. For example, the image may be segmented to extract the airways from the rest of the image as shown in FIG. 2. Then a path is planned including a series of shapes at 303. As described in prior path planning applications, this requires defining a seed location to start the search. Subsequently, a concentric cannula device is built to achieve the specified shapes, which is received by the practitioner at 304. Finally, a desired procedure may be performed on the patient at 305 by extending the tubes in the order specified.

Given the flexibility of modern technology, many of these operations may be performed remotely. For instance, data may be processed into a model of the interior space (e.g. segmented) in one location. A path through the space and a device suitable for following that path may be planned in a second location. Then the device may be assembled in a third location, before being returned to the technician or physician for insertion into the patient. Preferably, assembly of the nested cannula device will be performed in a manufacturing facility with good quality and sanitary controls; nevertheless, it might be that all these steps could be performed in a single location with the physician herself assembling the device to be inserted.

It has been proposed to use A* style path planning to facilitate deployment of active cannulas, see e.g. “3D TOOL PATH PLANNING, SIMULATION AND CONTROL SYSTEM,” prior, co-pending US application Ser. No. 12/088,870 of Trovato et al., filed Oct. 6, 2006, U.S. Patent Application Publication no. 2008/0234700, Sep. 25, 2008, which is incorporated by reference herein and made a part of this application. This type of planning makes use of a “configuration space.” A “configuration space” is a data structure stored on at least one machine readable medium. The configuration space represents information about a physical task space. In this case, the physical task space is the interior structure of the patient's body into which the active cannulas are to be inserted. The configuration space includes many “nodes” or “states,” each representing a configuration of the device during insertion.

FIG. 4 shows source program code for creating a node in a configuration space as taught by rior, co-pending U.S. provisional application Nos. 61/075,886, Jun. 26, 2008 and 61/099,223, Sep. 23, 2008, of Trovato et al., preferably improved to minimize memory using the method taught therein. Such program code is converted to machine executable code and embodied on a medium for use by the invention. When the code is executed, it will give rise to the configuration space data structure as embodied on a medium. This particular code has been found to be advantageous with respect to interior spaces of the human body. This code allows a 6D space to be compressed into 3D, by augmenting 3D configuration space paths, with high precision locations and orientations rather than inferring them from their configuration state position.

A* or ‘cost wave propagation,’ when applied to the configuration space, will search the configuration space, leaving directions, such as a pointer, leading to the ‘best path to the seed’ at every visited state. “Propagation of cost waves” involves starting from a search seed, often a target point. Propagation of cost waves through the configuration space data structure makes use of an additional type of data structure embodied on a medium known as a “neighborhood.” The neighborhood is a machine-readable representation of permissible transitions from one state in the configuration space to other states within the configuration space. For example in FIG. 6, a single curvature of a single arc (also called a fiber) is shown at eight evenly spaced rotations relative to a given location. The lengths of the arcs might be limited to less than 90 or 180 degrees depending upon the application, and the thread shown in the center (zero curvature arc) might also be limited to approximately the same length.

Propagation of cost waves also involves a “metric,” which is a function that evaluates the cost incurred due to transitioning from one state to a neighboring state.

The term “concentric cannulas” will be used herein to include Active Cannulas and Nested Cannulas, as described above. The present invention is applicable to both types.

An advantageous material for use in Active Cannulas is Ni—Ti alloy (nitinol). Nitinol has “memory shape”, i.e. the shape of a nitinol tube/wire can be programmed or preset at high temperatures. Therefore, at lower temperatures (e.g. room or body temperature) if a smaller tube extends from a larger one, it returns to its ‘programmed shape’. Another advantage of nitinol is that it can be used within an MRI machine. It is a relatively strong material and therefore can be made thin walled, enabling the nesting of several tubes. Tubes with an outer diameter from 5 mm down to 0.2 mm of 0.8 mm and below are readily available in the market. Other materials, such as polycarbonate may also be used, particularly for low cost, interlocking Nested Cannulas.

Concentric cannulas in accordance with the state of the art came in two general types:
“Active cannulas” meaning that tubes could rotate with respect to one another during employment; and
“Nested cannulas” meaning that tubes are to be constructed so as to prevent rotation as much as possible.

The present invention is applicable to both types. Preferably, though, the angular orientation of the tubes remains fixed throughout deployment as rotation can cause tissue damage.

An advantageous material for use in active cannulas is Ni—Ti alloy (nitinol). Nitinol has “memory shape”, i.e. the shape of a nitinol tube/wire can be programmed or preset at high temperatures. Therefore, at lower temperatures (e.g. room temperature) if a smaller tube extends from a larger one, it assumes its programmed shape. Another advantage of nitinol is that it can be used within an MRI machine. It is a relatively strong material and therefore can be made thin walled, enabling the nesting of several tubes. Tubes with an outer diameter from 5 mm down to 0.2 mm of 0.8 mm and below are readily available in the market. Nevertheless, other materials, such as various sorts of plastics might also be used.

The result of planning is preferably
a deployable physical set of concentric cannulas; and/or
a specification of the set of cannulas in terms of length and radius.

Certain areas for improvement remain with respect to the existing method and apparatus, e.g. better dexterity of the nested cannulas could be achieved if more radii of curvature could be used. Trauma to patient tissues could be further reduced by taking into account combined curvature affecting properties of several cannulas in the set.

It is desirable to achieve one or more of these goals using adaptive neighborhoods and improved calculations. Various objects and embodiments will become apparent in the rest of the text.

The following figures illustrate the invention by way of non-limiting example.

FIG. 1 shows a patient being scanned.

FIG. 2 shows a model of a lung.

FIG. 3 is a schematic flow diagram of the process in which the invention is to operate.

FIG. 4 shows example program code for implementing the key configuration space data structure elements.

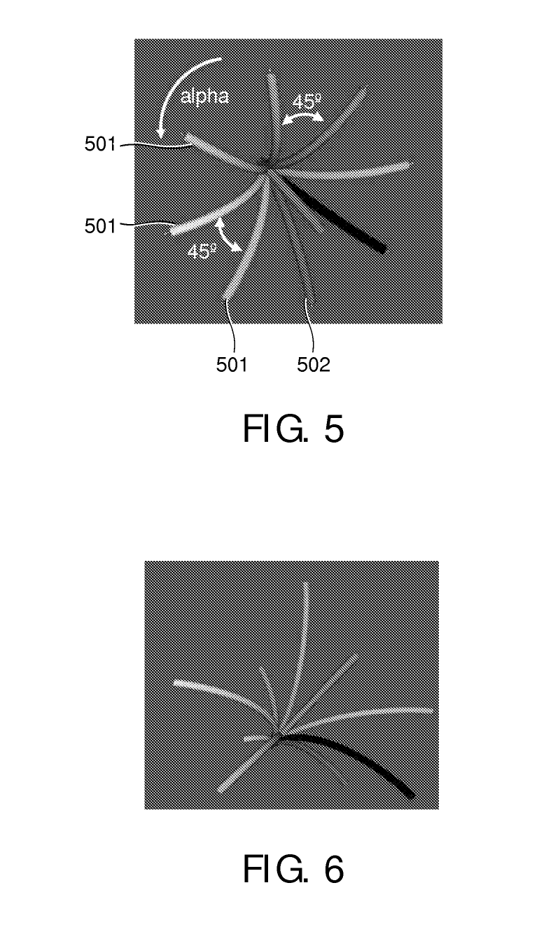

FIG. 5 is a schematic of a neighborhood representation of tube angular orientation choices showing symmetric angular orientations.

FIG. 6 is a schematic of a neighborhood representation of tube threads based on the interaction of the prior (parent) thread with the other nominal threads angled as shown in FIG. 5.

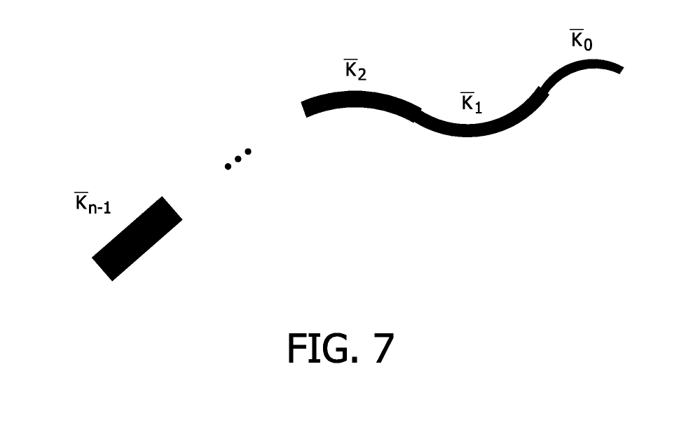

FIG. 7 is a picture of cannulas with various radii of curvature.

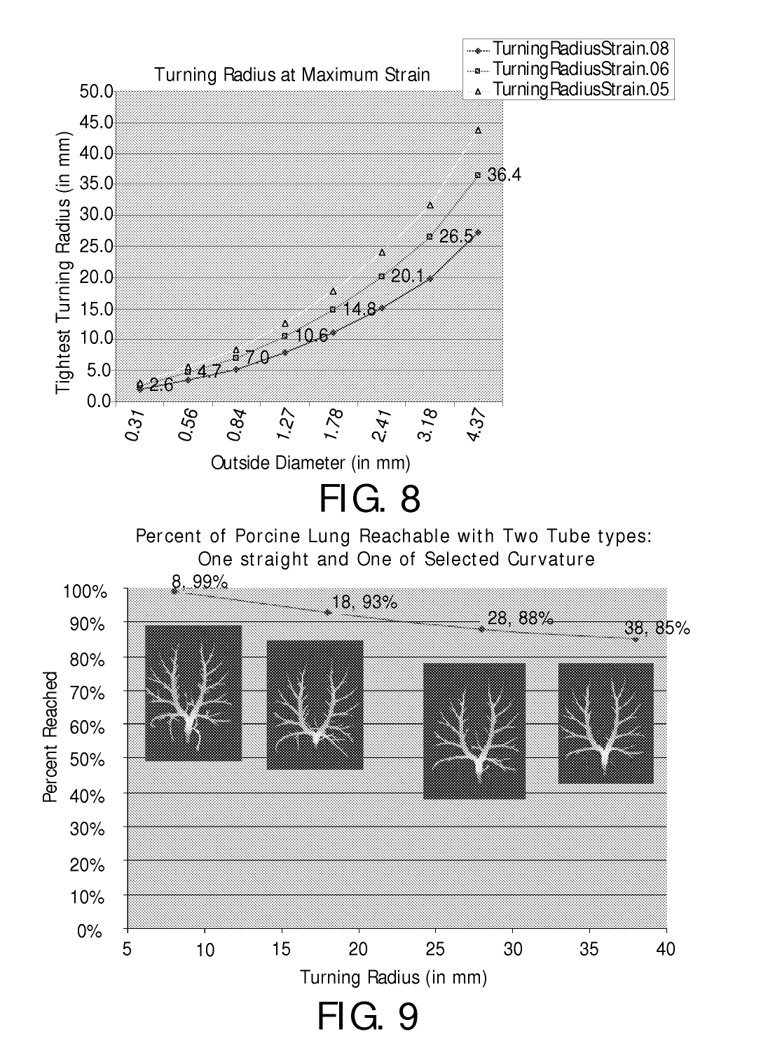

FIG. 8 is graph of simulation results relating to turning radius of a cannula as a function of maximum strain.

FIG. 9 is a graph of simulation results relating to percentage of a porcine lung reached as a function of cannula turning radius.

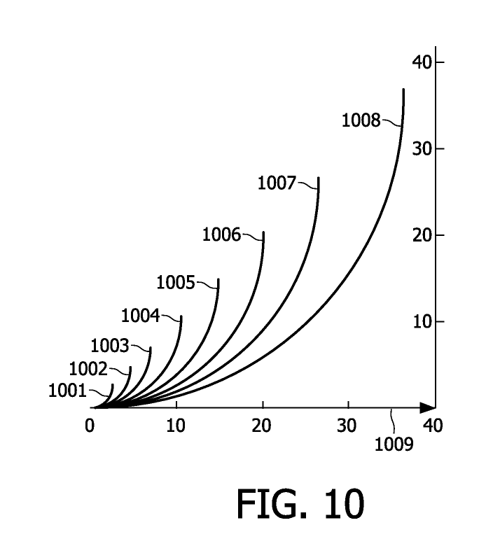

FIG. 10 illustrates the centerlines of a set of curved tubes.

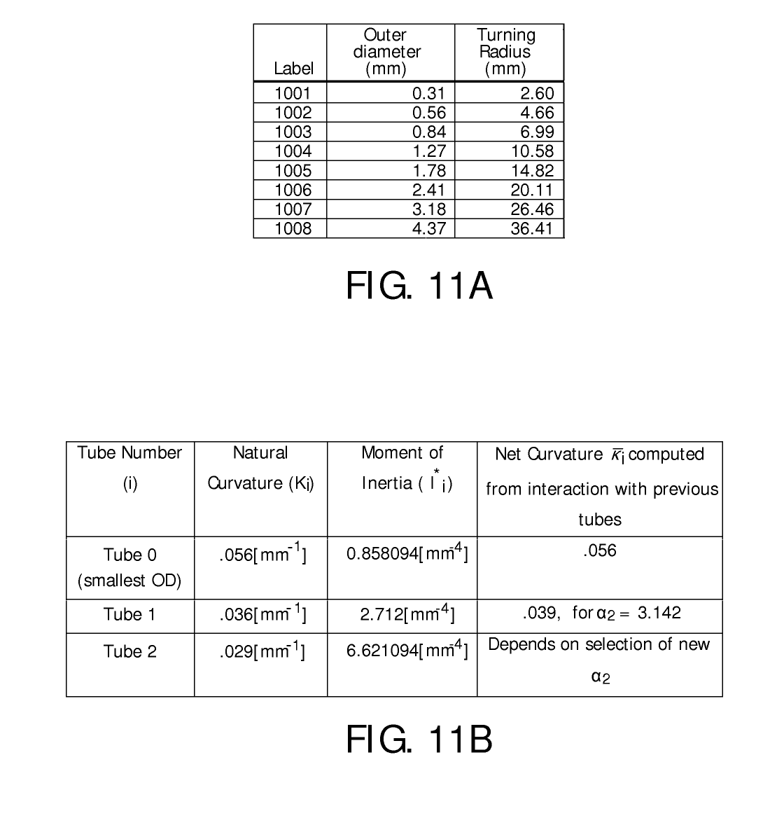

FIG. 11A is a table of radii and tube size for the tubes in FIG. 10

FIG. 11B is a table showing calculations of net curvature.

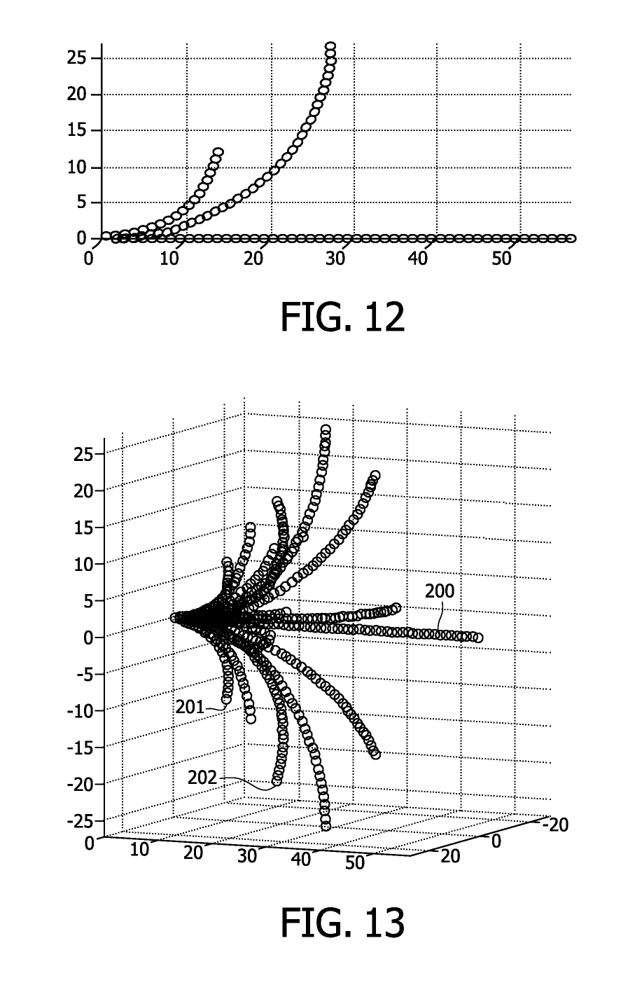

FIG. 12 shows three cannulas with three possible radii of curvature.

FIG. 13 shows a neighborhood resulting from rotations of the three cannulas in FIG. 12.

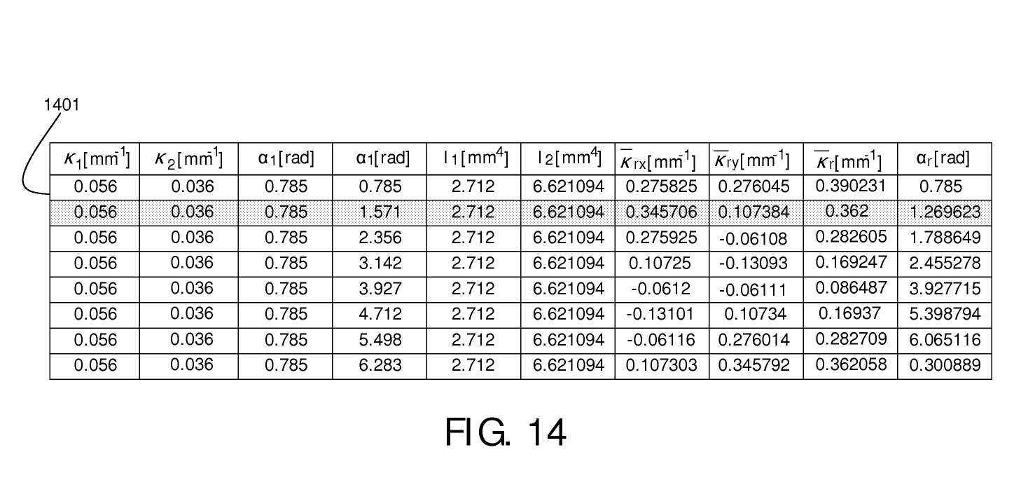

FIG. 14 is a table showing example input specifications for two tubes set at different orientations and example output specifications for the net curvature resulting from each pair of interactions, together forming a neighborhood.

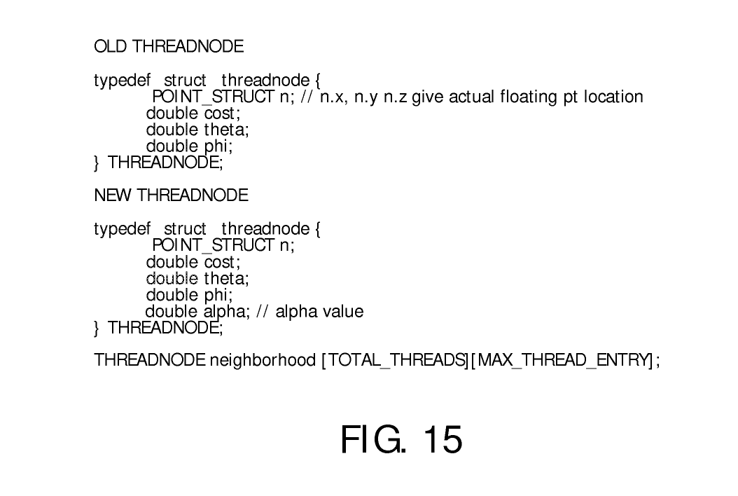

FIG. 15 is program code for implementing a neighborhood data structure element.

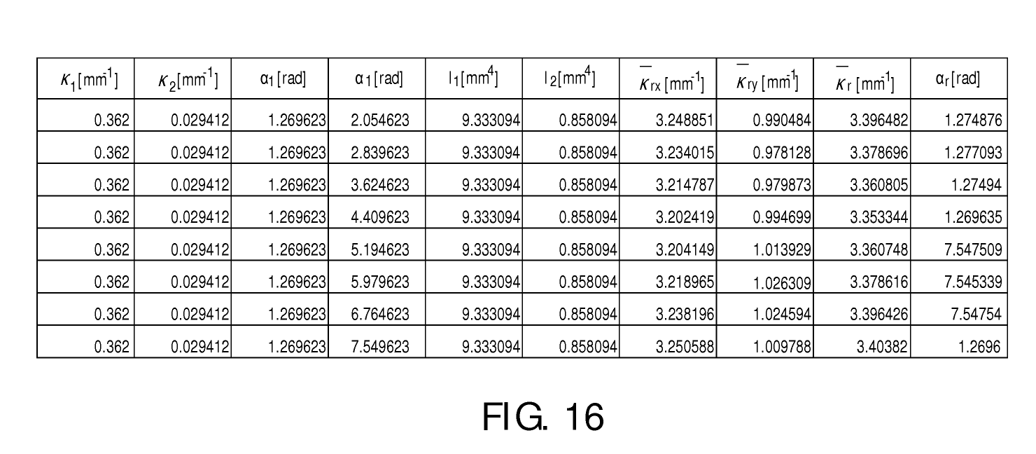

FIG. 16 is a table showing example input specifications for two tubes set at different orientations and example output specifications for the net curvature resulting from each pair of interactions, together forming a neighborhood.

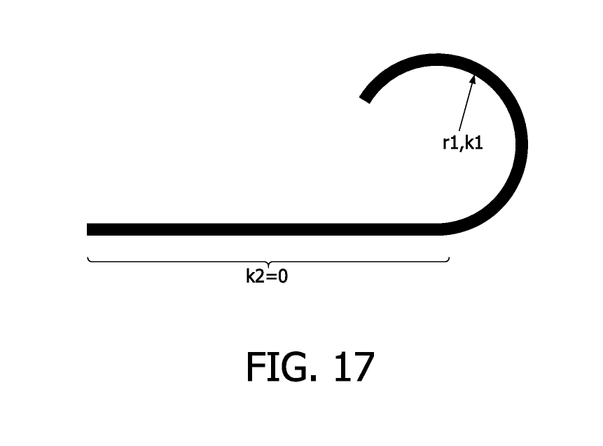

FIG. 17 is a schematic of a single tube with more than one curvature.

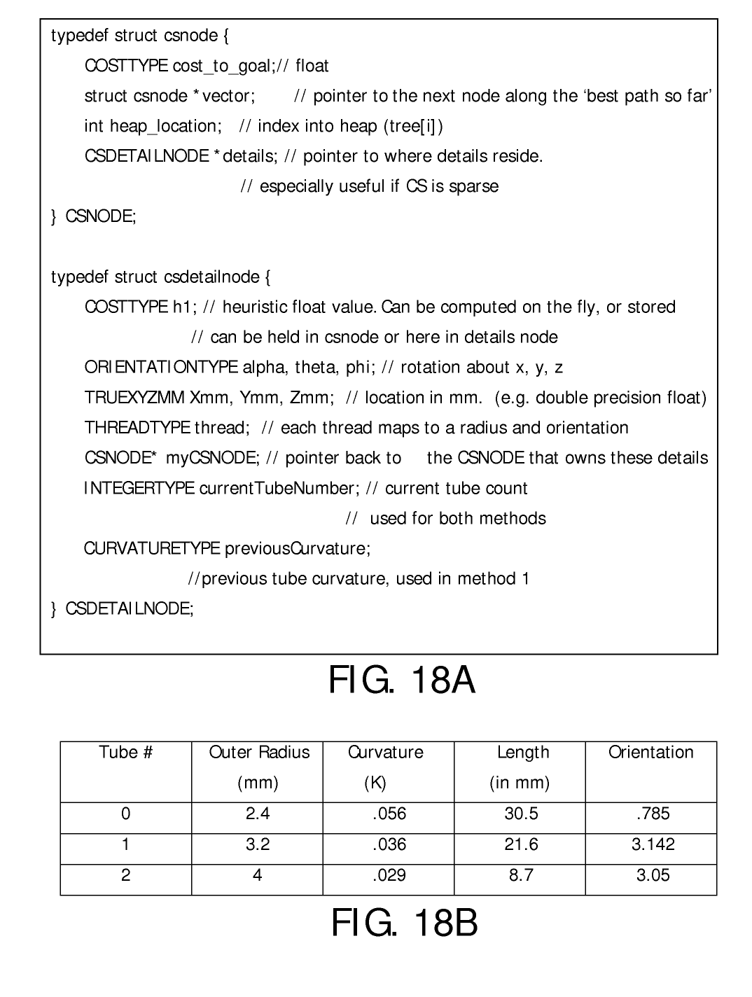

FIG. 18A shows example program code for implementing the revised key configuration space data structure elements.

FIG. 18B is a table showing examples of tube specifications.

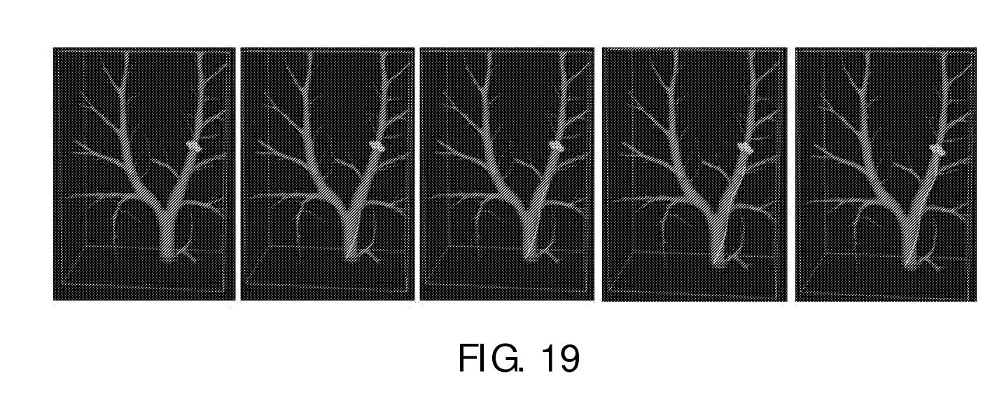

FIG. 19 shows an animation of tubes as they are extended one from another toward a target.

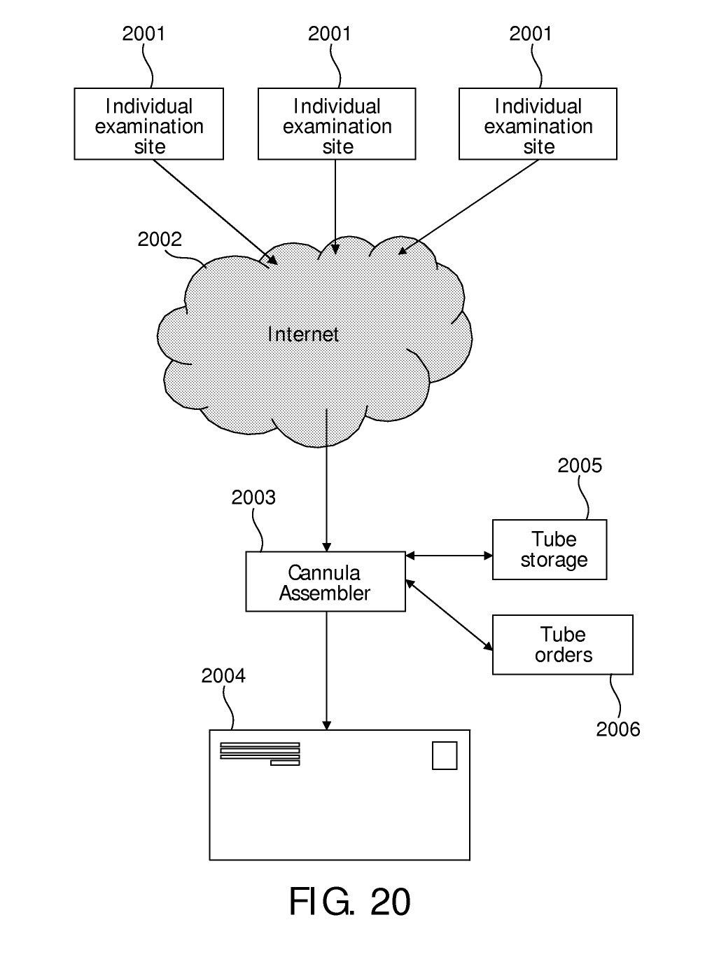

FIG. 20 shows a schematic of a manufacturing method.

Herein, the terms “tube” and “cannula” will be used interchangeably to refer to components of the device to be deployed. The terms “goal” and “target” will also be used interchangeably.

The smallest tube in a concentric set of tubes will be the central tube. This smallest tube will also be referred to herein as the “most distal” tube, since in typical use it can extend farther than the others. Similarly, the largest tube in the set is on the outside of the concentric set and will be referred to as the “most proximal” tube. This terminology expresses that, once the tubes are deployed, the largest tube will end closest to the point of insertion. The smallest tube will extend from the point of insertion, through an entire path, to a goal.

The fields of applicability of the invention are envisioned to include many types of procedures including imaging, chemotherapy, chemoembolization, radiation seeds, and photodynamic therapy, neurosurgery, ablation, laparoscopy, vascular surgery, and cardiac surgery. Concentric cannulas in accordance with the invention might be applicable to other situations, such as exploring the interior of a complex machine. Generalized versions of adaptive neighborhoods described herein may have applicability in the broader field of robotics.

It is desirable that a number of interdependent factors should be considered in planning the concentric cannula device and path. These include:
the site of a procedure or observation to be performed—this site also being known as a target;
the physical structure, including free space and obstacles that must be avoided;
the set of cannulas to be deployed;
the elasticity of each cannula;
the moment of inertia of each cannula; and
the radius curvature of each cannula.

### Interaction of Cannulas

A model of how cannulas interact mechanically with each other is found in the Webster et al. article cited below in the Bibliography. This article explains that concentric cannulas have curvatures that are a result of combined effects of all the cannulas.

As active cannulas rotate with respect to each other, both their joint curvature and curvature plane are changing. Therefore, the cannulas perform two movements: tip movement and lateral movement of the device. Whereas tip advancement is a desired feature, lateral movement of the body of the device might cause a collision with obstacles.

One approach to planning with various cannula elasticities is to make a full inverse kinematic model. Such a model may be advantageous in terms of elegance, but may also involve difficulties in terms of obstacle avoidance.

Another approach is to perform elasticity related calculations—after the path and a set of nested cannulas are planned—as a post hoc correction. This is discussed in co-pending application applicants's docket no. 012139US1, filed concurrently herewith.

A third approach is to use curvature affecting properties of the tubes as part of an artificial intelligence type search algorithm for path planning. An example of this is searching a configuration space using A* and an appropriately selected neighborhood, for instance as discussed below.

A description of a set of concentric cannulas can take the form of n sets of {κᵢ, αᵢ, Iᵢ}, where κᵢ is the curvature of the i-th segment, αᵢ is the angular orientation of i-th tube with respect to tube i−1, and is moment of inertia of the i-th tube cross-section.

Some example angular orientations of curved segments (sometimes called threads) are shown in FIG. 5 at 501, along with a straight segment 502 in the center. In the figure, the angle each value of αᵢ increases by an additional 45 degrees, in this case evenly dividing the full 360 degrees into 8 evenly spaced and symmetric curves. Each tube i is shown as being measured counter clockwise in the plane of the figure. The discretization chosen is for eight different angles, a symmetric set, with 45° between adjacent curves. The skilled artisan might choose more or fewer angles as required for a desired level of precision, and may alternatively choose angles that are not evenly spaced. More angles produce a more precise plan, but increase memory needs slightly and increase computational complexity. The skilled artisan must balance these factors in choosing the discretization. Preferably, the angular orientation αᵢ is fixed relative to the prior, i.e. proximal, tube. This ensures that the body of the overall device does not move laterally during deployment, ensuring obstacles are not touched—also called collision avoidance—and tissue trauma is reduced.

FIG. 6, which shows assymetric tube orientation choices, will be discussed further below.

FIG. 7 shows deployed concentric cannulas having various ‘net’ curvatures, κ̅₀, κ̅₁ . . . κ̅ₙ₋₁ in accordance with the invention. No tube is straight. Generally, smaller cannulas have the possibility of forming smaller turning radii (or equivalently, larger curvature) than the larger cannulas. This will be discussed in more detail later. Because this is a planar drawing, the deployed device is shown within a plane. In reality, the deployed device will have a three dimensional shape, in which various curvatures are in different planes.

### Selection of Tube Curvatures (κᵢ)

A tube i can achieve a minimum ‘turning radius’, or equivalently, a maximum curvature Kᵢ, where Rᵢ=1/Kᵢ, which depends upon the maximum strain value for the tube. This maximum strain is a property of the material. It is desirable to ensure that the tubes maintain their elastic ability to return to their original shape after they are extended from an enclosing tube, and to ensure that multiple manipulations are possible. These factors reduce the acceptable amount of strain further. The achievable curvature is a function of the tube's outer diameter and the strain. As described in FIG. 8, a smaller turning radius is possible with a smaller tube. Prior planning methods, however, assumed that all tube curvatures were equal to a single ‘turning radius’ of the largest tube. This assumption unnecessarily limited the dexterity of the cannulas of smaller diameter.

FIG. 8 show examples of minimum turning radius achievable at different strains based on outer diameter. The strains given are typical ones for nitinol. The horizontal axis shows the outside diameter of the tube expressed in millimeters, ranging from 0.31 to 4.37. The vertical axis shows the tightest (smallest) turning radius possible. The vertical axis is marked in millimeters, ranging from 0 to 50. The triangular points represent the turning radius as a function of outside diameter for tubes having a maximal strain of 0.05. The square points represent the turning radius as a function of outside diameter for tubes having a maximal strain of 0.06. The diamond points represent the turning radius as a function of outside diameter for tubes having a maximal strain of 0.08. As the outside diameter increases, the turning radius also increases.

In general, the maximum curvature, i.e. minimum radius of curvature, of each tube can be calculated in accordance with following equation:

$${κ}_{i}^{ma x} = \frac{2 {ɛ}_{i}}{{d}_{i}}$$

where dᵢ is the outer diameter of i-th tube, εᵢ is the strain of i-th tube (for tubes having the same material, ε will be the same for all tubes), κᵢ^{max} is the maximum curvature, see the Webster article cited below in the bibliography at p. 2858

FIG. 9 shows a study of reachability, based on a neighborhood, such as the one shown in FIG. 5, having eight curved neighbors and one straight neighbor. The curved neighbor is assumed to have a fixed turning radius for all diameter tubes. The horizontal axis is the selected turning radius in millimeters ranging from 5 to 40. The vertical axis shows the percentage of all airway voxels reached, ranging from zero to 100%. In this figure, points are highlighted for 8 millimeter turning radius, in which 99% of a sample porcine lung can be reached; 18 millimeters in which 93% of the porcine lung can be reached, 28 millimeters in which 88% of the porcine lung can be reached, and 38 mm, in which only 85% of the lung can be reached. The lung model illustrations superimposed on the curve show how increasing radius of curvature reduces access to fine structure in the lungs. There is an illustration for each point on the graph.

For the purpose of this simulation, based on a computer model of a porcine lung, the diameter of the tube was not considered as a limitation from a collision viewpoint. During path planning calculations, as long as the path lay within the lung, the point was considered theoretically ‘reachable’ by wider tubes—absent the turning radius issues.

While simulations with respect to the effect of turning radius on reachability are given here with respect to a lung, advantageous results could be achieved with respect to other body areas, such as vasculature.

By customizing a path planner in accordance with the incorporated patent documents to use, as an option, the tightest possible curvature for each size of tube, sets of concentric cannulas of greater dexterity can be planned. However, the curvature of i-th tube might be selected to have any value in the allowed range κᵢ∈[0,κᵢ^{max}] where zero curvature defines a straight tube.

FIG. 10 shows an example set of curvatures that extend until the tip is rotated 90 degrees, plus a straight thread shown on the same graph. These curvatures indicate the centerline of the tube, but do not indicate the inner or outer diameter. The threads shown have example curvatures related to the outer diameter, as set forth in the table of FIG. 11, with the straight thread 1009. A selected curve can be rotated about the X-axis to form a set of related curved thread choices that can be represented in the neighborhood data structure. FIG. 12 shows an example of a set of three curvature choices. FIG. 13 shows the three curvatures being rotated to several discrete angular orientations to create a schematic illustration of a neighborhood of tube choices.

As the set of concentric cannulas is assembled, the number of the cannula defines the outermost diameter of the tube. The number of the tube will be accompanied by a specific curvature of the tube and orientation, which results from the neighbor selected by the planner.

### Adaptive Neighborhood

An “adaptive” neighborhood is one that can change as a function of state in the configuration space. These changes will usually be based on values associated with one or more adjacent states and usually occur dynamically during cost wave propagation. In the case of concentric cannulas, the neighborhood will change as a function of tube number, and also as a function of tubes previously visited during cost wave propagation.

If two tubes with different curvatures are angularly rotated with respect to each other, their angular and curvature interaction has to be considered. The resulting curvature has two planar components. The generalized form of the resulting ‘net curvature’ relies on the elastic interaction between two tubes, and is given as:

$${{κ}^{\overline{\to }}}_{r} = \frac{1}{{E}_{1} {I}_{1} + {E}_{2} {I}_{2}} \cdot [ \begin{matrix}{E}_{1} {I}_{1} \cdot {κ}_{1} \cdot sin {α}_{1} + {E}_{2} {I}_{2} \cdot {κ}_{2} \cdot sin {α}_{2} \\ {E}_{1} {I}_{1} \cdot {κ}_{1} \cdot cos {α}_{1} + {E}_{2} {I}_{2} \cdot {κ}_{2} \cdot cos {α}_{2}\end{matrix} ] \qquad ( 1 )$$

where α₁ and α₂ are rotation angles around a reference axis, κ₁ and κ₂ are curvatures of tubes, and E₁I₁ and E₂I₂ are the products of the Young's modulus and moment of inertia for each tube, respectively.

The resulting curvature ( \overline{\vec{κ}}ᵣ) is a 2D vector:

$${{κ}^{\overline{\to }}}_{r} = [ \begin{matrix}{\overline{κ}}_{rx} \\ {\overline{κ}}_{ry}\end{matrix} ] , \qquad ( 2 )$$

with [symbol] and [symbol] =tan⁻¹(κᵣₓ/κ_{ry}) as the final (net) curvature and orientation. Values { κ̅ᵢ, α̅ᵢ} represent net curvatures and net orientations for the tube being assembled whereas values {κᵢ,αᵢ} represent the ‘natural’, pre-assembled curvatures and orientation of tubes.

During deployment, there will typically be an interaction between more than two tubes, e.g. three tubes with moments Iᵢ, Iᵢ₊₁, Iᵢ₊₂, curvatures κᵢ, κᵢ₊₁, κᵢ₊₂, and angles αᵢ, αᵢ₊₁, αᵢ₊₂, where i represents the outermost tube. To simplify computation, the resulting curvature will be computed using Eq. (1) using the fact that two nested tubes (e.g. i and i+1), act as one tube when interacting with a third tube. The two have the moment of inertia I₁=Iᵢ+Iᵢ₊₁, and curvature κ₁= κ̅ᵢ. The third tube acts as one tube, defining I₂=Iᵢ₊₂ and κ₂=κᵢ₊₂.

If this adaptive neighborhood model is used to compute the threads of the ‘neighborhood of permissible motions’ in U.S. Provisional No. 60/725,185 (WO 2007/042986), then in each propagation step it is possible to account for tube interaction as A* algorithm propagates. The propagation preferably occurs from the ‘seed’ located where the smallest tube will extend, typically to the target, so that tubes can be layered sequentially.

To simplify calculation herein, it will be assumed that all the tubes are made of the exactly same material, so that Eᵢ=E, ∀i and Iᵢ=const₁·(rₒ⁴−rᵢ⁴), where rₒ⁴ and rᵢ⁴, are outer and inner radius of the tube, respectively, and const1 is a constant number, with

$${const}_{1} = \frac{π}{64} .$$

In this case, Eq. (1) can be simplified cancelling out all Eᵢ and const₁, thus using Iᵢ*=Iᵢ/const₁. The skilled artisan might alter the device to include different materials. In such a case, the calculation would have to be altered to reflect that.

FIG. 11B shows an example using 3 tubes, numbered 0, 1 and 2:

Tube 0 is the smallest tube, having the smallest outer diameter (OD), which typically contacts the target (seed location for the search). Tube 0 has a turning radius of 18 mm (curvature=0.056 mm⁻¹) and angular orientation of 45° (0.7854 rad). Tube 1 is the next physical tube, which has a curvature 0.036 mm⁻¹ (radius=28 mm). Tube 2 has a curvature of 0.029 mm⁻¹.

From the interaction model of Equation (1) for Tube 0 and Tube 1, it is possible to compute the resultant interactions to define net curvatures and angles. This is shown in the table of FIG. 14. This table is therefore an example of two interacting tubes, the first tube is assumed to be set at some angle, for example it might be the angle of the tube leading from the target to the current location. The second tube may be positioned in any of 8 different angles, extending from the tip of the first for example. The net result of their interaction is a net neighborhood, similar to that shown in FIG. 6. Therefore, instead of a symmetric neighborhood as in FIG. 5, the net neighborhood is asymmetric, both in curvature and angle as shown in FIG. 6 and it describes the path that would be taken as a result of the interacting tubes.

The asymmetry can be observed from the table of FIG. 14, in the last two columns. This neighborhood is not computed only once, but has to be recomputed at each segment transition, which is typically when each new node is opened (expanded). Segments and segment transitions are shown in FIG. 7, where, for the first (smallest) tube, subscript 0 (zero), arc shaped threads in the neighborhood are uniformly distributed on a 360° circle (for example at every 45° for the 8-arced neighborhood of FIG. 5). Therefore it is possible to compute the rotational angle from an arc index i (alpha=(i−1)*45°, where i is the current thread number). For each subsequent tube (subscripts 1 to n−1), an adaptive neighborhood must be computed. Given the complexity of tube interaction, the value for angle has been added to the neighborhood in FIG. 14.

FIG. 15 shows program code for implementing a neighborhood and data structure element. It should be noted that in prior applications, a neighborhood was sometimes called a ‘brush’ (because it looks like one).

In the example of FIG. 15, the earlier method, labeled “OLD THREADNODE”, could use the thread number to derive the angle (alpha), since it is the first index of the neighborhood. In the example of FIG. 15, the currently proposed method, labeled “NEW THREADNODE”, explicitly stores the computed (net) alpha value since it is dependent upon the prior tubes and will be used to compute subsequent tube interactions.

In FIG. 15, n represents a point on the neighborhood, described by the actual (x, y, z in mm for example) position, theta and phi are orientations of the neighborhood in the configuration space coordinate space and alpha is the orientation of a neighborhood thread relative to previous tube.

In the THREADNODEs of the neighborhood data structure, the values of the variables alpha, n, cost, theta and phi change as the algorithm propagates.

However, it is necessary to maintain the orientation between segments of the nested tubes, for example, to be able to use interlocking mechanisms as proposed in prior, co-pending U.S. provisional application No. 61/106,287 of Greenblatt et al., filed Oct. 17, 2008. This ensures that the angle inserted at one end of the tube remains consistently oriented throughout the tube. If the interlock used is a hexagonal shape for example, and tubes are bent along the flat faces, then a second tube could be conveniently locked into any one of the 6 possible orientations. The neighborhood of ‘net threads’ would then be computed based on the current tube curvature and interactions with six arc-shaped tubes, and optionally a straight tube.

A net neighborhood is therefore computed based on the first tube and some number of possible orientations for physical tubes to be set. These orientations might be evenly distributed, or may be unevenly or preferentially clustered in certain directions, depending upon the application. For example, it may be preferable to avoid tubes that lead in the exact opposite direction from the current tube.

To achieve evenly distributed angles covering the full rotation, assuring a fixed discrete relative angle between the segments, n threads are computed with a nominal 2π/n angle between them. Assuming there is interaction between the tubes, resulting angles α̅ will not be uniformly distributed, however.

An example adaptive neighborhood is computed next, using the above set of tubes (Tube0, Tube1 and Tube2), and assuming they have an octagonal cross section. The planner creates the neighborhood when the lowest cost node is ‘opened’. In our example, we will assume the ‘best path so far’ has followed an arc marked 1401 in FIG. 14. The next neighborhood should be computed using 8 uniformly distributed angles, starting from the previous angle. An example of how to build a neighborhood from the segment marked with a shaded box in FIG. 14, having net curvature 0.039 mm⁻¹ and having a resulting orientation of 1.48 radians, is given in the table of FIG. 16. The net curvature 0.039 mm⁻¹ representing the overall (net) interaction of tube 0 and tube 1 κᵣ in FIG. 14 becomes κ₁ in FIG. 16. The resulting orientation αᵣ=1.480 in FIG. 14 becomes α₁ in FIG. 16. In this table, α₂ is uniformly distributed starting at α₁ in π/4 (=0.785) steps. Clearly, the neighborhood could be distributed in other patterns, however for a hex shaped tube such as is described in this example, it is preferable to choose angles in increments aligned relative to the faces.

As the algorithm explores the space of possible tube sets within the configuration space, which might also be thought of as an obstacle space, the neighborhood is being adapted to the best tube series used ‘so far’. The computation of an adaptive neighborhood uses the previous net curvature (0.039), the previous net angle (1.48 radians), the previous moment of inertia, the next tube's curvature (0.029), and the next tube's moment of inertia to compute the next adapted neighborhood.

FIG. 18A shows example program code for implementing revised configuration space data structure elements to be used when taking into account curvature affecting properties in path planning. Since the curvatures for each tube number (each tube size) are known prior to planning, the curvature of the ‘next tube’ can be detected using next tube's tube number (currentTubeNumber, FIG. 18A) from a lookup array of tube curvatures. Therefore, the node structure as described in U.S. Provisional 61/075,886 and FIG. 4 is preferably extended by a variable, currentTubeNumber, as in FIG. 18A. Since diameters of each tube number are known prior to planning, each tube's moment of inertia can be pre-computed and stored in a look-up array.

There are two different approaches to obtain the parent's parameters, i.e. curvature ( k̅ᵢ₋₁) and angle ( α̅ᵢ₋₁) for the i-th node.

### Method 1:

In this method, a node structure as described in U.S. Provisional 61/075,886 and FIG. 18A is further extended with a variable, previousCurvature. This variable is updated whenever a node is ‘improved’, so that it stores the net curvature ( k̅ᵢ₋₁) used to arrive at this node. Therefore one computation—as described in Eq. (1) and (2) and FIG. 16—is performed for each thread during a new expansion. In a preferred embodiment, as threads are explored, if the current thread is the same as the parent's thread, then the curvature, angle and tube number can be kept from the parent. This essentially allows the parent's tube to be extended, without adding another tube. If the time to compute interaction, from Eq. (1) and (2), is t, the computation time of this approach for one expansion is O(N*t), where N is the number of threads in the neighborhood. This method would increase the memory needed to solve the problem by M*sizeof(CURVATURETYPE), where M is the total number of improved nodes and sizeof(CURVATURETYPE)=4 bytes on a 32-bit processor. However, using the reduction of memory methods as described in U.S. Provisional 61/075,886, this is a small increase in configuration space size.

### Method 2:

In this method, the curvature of previous parent is not saved, but rather computed ‘on the fly’. The interaction is computed by extracting the characteristics of all prior tubes, starting with the pointer to the parent, the pointer being the variable vector, FIG. 18A. Since pointers can be used to trace back to the seed point, it is possible to retrieve the physical curvature of each tube and relative rotation with respect to previous tube:

αᵢ=(thread−1)·2·π/(N−1),

where N is the number of threads in the neighborhood.

Using Equations 1 and 2 iteratively for each segment, the net curvature to reach the i-th node can be computed.

This method does not require more memory than described in U.S. Provisional 61/075,886. The computation time at each expansion is O((N+currentTubeNumber−1)*t), which is an increase of O((currentTubeNumber−1)*t) for each expansion.

In some cases, it may be easier to manufacture tubes that contain both a straight portion and a curved portion, as shown in FIG. 17. This example tube has a straight portion with curvature K₂=0 and a curved portion having a radius of curvature=2 cm, or equivalently, K₁=0.5. Such a tube might be treated as two tubes in the calculations above, with each tube having the same moment of inertia and Young's modulus, but different curvatures. Also, the calculation has to be adjusted to show that these “two” tubes do not interact with each other.

Typically, in an assembly with more than three tubes, the innermost tubes will stop having a significant effect on the total curvature of the device in areas where there are more than 3 overlapping tubes. The calculation may be simplified by applying a threshold to determine how many tubes are considered to contribute to a net curvature. One type of threshold might relate to determining when an inner tube has a moment of inertia that is less than some predetermined percentage of the moment of inertia of some outer tube. One such predetermined percentage might be 10%. Another threshold might be to consider, in the region of overlap, only a predetermined number of outer tubes, such as three.

The result of the preceding calculations is a tube set specification, typically in the form of a list of tubes with sequence numbers. Each sequence numbered tube will also specify a diameter, curvature, length, and orientation: for example as shown in FIG. 18B.

The output may be in the form of an animation or some other graphic output. FIG. 19 shows such an animation, where the sequential frames illustrate a concentric cannula advancing in the lung. Such an animation might be accompanied by audio or text instructions relating to tube characteristics and/or deployment of the tubes. A manufacturer, upon receiving the specification, will produce a device including a set of concentric cannulas. The cannulas will preferably be shipped in an airtight, sterile packaging arranged with their distal ends flush. Deployment will preferably be by inserting the assembly and then advancing the inner tubes in reverse order of tube diameter, until the assembly has all the proximal ends of the tubes flush. Other orders are possible and might entail different types of tube interactions.

FIG. 20 shows schematically a number of individual examination sites 2001 providing examination data over the internet 2002 to an assembler of sets of cannulas 2003, which in turn ships many assembled sets of concentric cannulas 2004 to appropriate clinics and hospitals where they can be deployed into patients. Generally, planning concentric cannula devices may start with a discrete set of pre-ordered and stored tubes 2005. This discrete set reduces manufacturing costs by reducing the number of tubes, especially the number of specific curvatures a manufacturer has to have in stock. Tubes also will have to be ordered at 2006, with variations possible as business needs change.

From reading the present disclosure, other modifications will be apparent to persons skilled in the art. Such modifications may involve other features which are already known in the design, manufacture and use of medical robotics and which may be used instead of or in addition to features already described herein. Although claims have been formulated in this application to particular combinations of features, it should be understood that the scope of the disclosure of the present application also includes any novel feature or novel combination of features disclosed herein either explicitly or implicitly or any generalization thereof, whether or not it mitigates any or all of the same technical problems as does the present invention. The applicants hereby give notice that new claims may be formulated to such features during the prosecution of the present application or any further application derived therefrom.

The word “comprising”, “comprise”, or “comprises” as used herein should not be viewed as excluding additional elements. The singular article “a” or “an” as used herein should not be viewed as excluding a plurality of elements. The word “or” should be construed as an inclusive or, in other words as “and/or”.

### BIBLIOGRAPHY

DE 4223897 C2
U.S. Pat. No. 6,572,593
R. J. Webster & N. J. Cowan, “Toward Active Cannulas: Miniature Snake-like Surgical Robots” 2006 IEEE/RSJ (October 2006, Beijing, China) pp. 2857-2863
K. I. Trovato, A* Planning in Discrete Configuration Spaces of Autonomous Systems, (U. of Amsterdam 1996)
U.S. Pat. No. 6,251,115
P Sears et al., “Inverse kinematics of concentric tube steerable needles”, IEEE Conf. on Robotics and Automations, pp. 1887-1892 (2007)

1. A computer method for configuring a device, the method comprising executing operations on at least one data processing device, the operations comprising:
receiving a representation of permissible tube specifications, the tube specifications comprising at least one indication of a diameter and at least one respective radius of curvature for each diameter, wherein for at least one tube, the respective radius of curvature is smaller than would be possible for at least one tube of larger diameter;
receiving a description of a space to be explored by the device, the description including representations of at least one start point,
at least one free space and/or at least one obstacle, and
at least one goal point;
selecting a plurality of tubes such that when the tubes are deployed, they will define at least one path through the space from the start point to the goal through free space and avoiding obstacles, such selecting being responsive to the set and description, an output of such selecting including:
a specification of at least one sequence of tubes;
at least one radius of curvature for each tube;
a respective length for each selected tube; and
an angular orientation of tube with respect to previous tube.

2. The method of claim 1, wherein at least one of the radii of curvature is chosen based on a tightest possible turning radius for the respective tube diameter.

3. The method of claim 1, wherein the representation corresponds to a discrete set of tubes expected to be maintained at a manufacturing facility.

4. The method of claim 1, wherein
the representation comprises a machine readable neighborhood data structure embodied on at least one medium;
the description of the space comprises a machine readable configuration space data structure embodied on the medium; and
the selecting comprises applying A* by propagating cost waves through the configuration space responsive to the neighborhood.

5. The method of claim 1, wherein the output of the selecting comprises an animation.

6. A computer method for planning a set of concentric tubes comprising executing operations in at least one data processing device, the operations comprising:
receiving a machine readable description of a space to be explored in the form of a machine readable configuration space data structure embodied on a medium and taking into account at least one start, at least one goal, and at least one obstacle;
receiving a machine readable adaptive neighborhood data structure embodied on a medium and comprising a representation of a plurality of tubes of different diameters and at least one respective curvature affecting property corresponding to each tube;
propagating cost waves from a most distal tube to a most proximal tube, using A*, the configuration space data structure, and the adaptive neighborhood data structure, avoiding the obstacle, such propagating including accumulating effects of individual tubes so that a net curvature of the set combines the effects of at least some of the respective curvature affecting properties; and
responsive to A*, outputting a specification of a set of tubes to be deployed to follow a path from the start to the goal resulting from propagation of cost waves.

7. The method of claim 6, wherein the curvature affecting property is radius of curvature.

8. The method of claim 6, wherein the curvature affecting property is moment of inertia.

9. The method of claim 6, wherein the curvature affecting property is elasticity.

10. The method of claim 6, wherein the curvature affecting property is angular orientation.

11. The method of claim 6, wherein accumulating comprises
saving a net curvature in at least one state node of the configuration space; and
using the saved net curvature in opening a next state node after the one state node.

12. The method of claim 6, wherein accumulating comprises
upon opening a current state node of the configuration space data structure, retrieving at least one value relating to at least one curvature affecting property corresponding to at least one individual tube selection made prior to such opening; and
combining the value with a value associated with a current individual tube selection.

13. The method of claim 6, wherein the neighborhood data structure comprises, for at least one tube, asymmetric resultant angular orientation values that accumulate effects of curvature affecting properties from at least one other tube.

14. The method of claim 6, wherein the neighborhood data structure is adaptive in that the neighborhood depends on tube number and comprises for each tube number
at least one respective value responsive to elasticity and/or moment of inertia;
a plurality of respective angular rotation values, which are symmetric for at least one tube number and asymmetric for at least one other tube number; and
an option for selecting a tube having a respective turning radius corresponding to a. respective diameter, such that the turning radius is smaller than would be possible for a more proximal tube.

15. The method of claim 6, wherein the output is an animation.

16. A computer method comprising executing operations on at least one data processing device, the method comprising:
maintaining a first representation of a physical task space to be penetrated;
maintaining a second representation of a set of tubes to be deployed in the space, the second representation including an indication of at least one curvature affecting property for each tube;
planning a configuration of concentric tubes, responsive to the first and second representations, while taking into account interactions between the tubes due to the curvature affecting property, the configuration indicating a net curvature of the configuration; and
communicating the configuration in a form from which an assembly of concentric tubes can be produced.

17. The method of claim 16, wherein
the first representation comprises a machine readable configuration space data structure embodied on a medium,
the second representation comprises a machine readable neighborhood data structure embodied on a medium, and
planning comprises applying A* to the configuration space responsive to the neighborhood taking into account the curvature affecting property.

18. The method of claim 17, wherein the neighborhood is adaptive.

19. An assembly of concentric tubes, assembled responsive to results as claimed in claim 1, and ready for deployment into a human body.

20. A medium readable by a data processing device and embodying a data structure, the data structure being a neighborhood for use in planning a path for and/or a configuration of a robotic device, such planning being in conjunction with cost wave propagation using A*, and a configuration space data structure, the neighborhood comprising a plurality of sub-data structures, each sub-data structure representing a possible choice along the path and/or in the configuration, wherein the neighborhood is adaptive.

21. The medium of claim 20, wherein one or more values and/or sub-data structures within the data structure is dependent on a previously selected option for path and/or device configuration.

22. The medium of claim 20, wherein
planning is for configuration of an assembly of concentric cannulas,
the configuration space comprises a representation responsive to a scan of an interior portion of a body into which the assembly is to be deployed,
each sub-data structure comprises a representation of a tube and a respective curvature affecting property of that tube; and
the neighborhood is adapted, in conjunction with A* and the configuration space, to output the assembly.

23. A system comprising:
an interface for receiving data relating to a scan of a body;
at least one medium for storing code and data;
at least one processor for planning a set of concentric cannula by performing operations, the operations comprising
receiving a representation of permissible tube specifications, the tube specifications comprising at least one indication of a diameter and at least one respective radius of curvature for each diameter, wherein for at least one tube, the respective radius of curvature is smaller than would be possible for at least one tube of larger diameter;
receiving a description of a space to be explored by the device, the description including representations of at least one start point,
at least one free space and/or at least one obstacle, and
at least one goal point;
selecting a plurality of tubes such that when the tubes are deployed, they will define at least one path through the space from the start point to the goal through free space and avoiding obstacles, such selecting being responsive to the set and description, an output of such selecting including:
a specification of at least one sequence of tubes;
at least one radius of curvature for each tube;
a respective length for each selected tube; and
an angular orientation of tube with respect to previous tube
an interface for supplying a specification of nested cannulas responsive to the planning.

24. A system comprising:
an interface for receiving data relating to a scan of a body;
at least one medium for storing code and data;
at least one processor for planning a set of concentric cannula by performing operations, the operations comprising
receiving a machine readable description of a space to be explored in the form of a machine readable configuration space data structure embodied on a medium and taking into account at least one start, at least one goal, and at least one obstacle;
receiving a machine readable adaptive neighborhood data structure embodied on a medium and comprising a representation of a plurality of tubes of different diameters and at least one respective curvature affecting property corresponding to each tube;
propagating cost waves from a most distal tube to a most proximal tube, using A*, the configuration space data structure, and the adaptive neighborhood data structure, avoiding the obstacle, such propagating including accumulating effects of individual tubes so that a net curvature of the set combines the effects of at least some of the respective curvature affecting properties; and
responsive to A*, outputting a specification of a set of tubes to be deployed to follow a path from the start to the goal resulting from propagation of cost waves.
an interface for supplying a specification of nested cannulas responsive to the planning.

25. A system comprising:
an interface for receiving data relating to a scan of a body;
at least one medium for storing code and data;
at least one processor for planning a set of concentric cannula by performing operations, the operations comprising
maintaining a first representation of a physical task space to be penetrated;
maintaining a second representation of a set of tubes to be deployed in the space, the second representation including an indication of at least one curvature affecting property for each tube;
planning a configuration of concentric tubes, responsive to the first and second representations, while taking into account interactions between the tubes due to the curvature affecting property, the configuration indicating a net curvature of the configuration; and
communicating the configuration in a form from which an assembly of concentric tubes can be produced;
an interface for supplying a specification of nested cannulas responsive to the planning.

26. A deployable set of concentric cannulas, the set comprising a plurality of tubes, each tube being defined by parameters including diameter, length, and turning radius, at least one of the plurality of tubes having a respective turning radius that is tighter than would be possible for a tube of larger diameter.

27. The set of concentric cannulas of claim 26, wherein the respective turning radius is a tightest possible value in view of the maximal strain for the one tube.

28. The set of concentric cannulas of claim 26, wherein at least first and second tubes are not straight and have first and second radii of curvature, respectively, the first and second radii of curvature not being equal.
